(19) United States
(12) Patent Application Publication
(10) Pub. No.: US 2008/0319397 A1
(43) Pub. Date: Dec. 25, 2008

(54) Holder for a stomach insert feeder tube

(75) Inventor: Ignazio Macaluso, Brooklyn, NY (US)

Correspondence Address: EZRA SUTTON, P.A., SUITE 201, PLAZA 9 BUILDING, 900 ROUTE 9 NORTH, WOODBRIDGE, NJ, 07095, US

(73) Assignee: JACK A. AINI

(21) Appl. No.: 11/821,252

(22) Filed: Jun. 21, 2007

(51) Int. Cl. A61M 25/02

(52) U.S. Cl. 604/174

(57) ABSTRACT

A holder for receiving a stomach insert feeding tube therein for use in the gastronomical feeding of a patient. The feeding tube holder includes a substantially donut-shaped housing having a passageway for receiving the feeding tube therethrough. The donut-shaped housing includes a channel connected to the passageway for allowing the removal of the feeding tube holder without the need for removing the feeding tube from the stomach wall of the patient. The donut-shaped housing also includes an outer flange member for contacting a pad positioned on the stomach surface of the patient. Additionally, the donut-shaped housing further includes an exterior circular side wall and an interior circular side wall being connected to each other by a top wall member. The exterior side wall has a greater height than the interior side wall; and the interior side wall is used for maintaining the passageway at an elevated position spaced above the stomach surface for maintaining the feeding tube at a fixed position above the stomach surface.

### FIELD OF THE INVENTION

The present invention relates to a holder for a stomach insert feeding tube for insertion of the insert feeding tube within a central opening of the holder. More particularly, the holder for the stomach insert feeding tube minimizes chaffing caused by movement of the feeding tube at the stomach penetration point, eliminates abrasions by the feeding tube, lessens the chance for infections, and also provides comfort to the patient.

### BACKGROUND OF THE INVENTION

The use of gastronomy feeding tubes, stomach feed tubes, and the like are well-known in the prior art. Typically, there is a holding structure for the gastronomy feeding tube so that feeding of the patient is directed through the feeding tube into the intestinal tract or stomach. Typically, a portion of the feeding tube projects from the patient for receiving food, nutrients or liquid substances when such food or drink cannot be consumed orally. These gastronomy feeding tube holders are used to transport and orient the feeding tube relative to the patient's stomach or intestinal tract. Many of these feeding tube holders are uncomfortable, infection prone and unyielding in use for the patient.

There remains a need for a holder for a stomach insert feeding tube that minimizes chaffing caused by movement of the feeding tube relative to the stomach penetration point. Additionally, the feeding tube holder would eliminate abrasions to the stomach by the feeding tube and decrease the chance of infection caused by the feeding tube. The structure and design of the feeding tube holder would also provide comfort to the patient when in an operational mode.

### DESCRIPTION OF THE PRIOR ART

Stomach insert feeding tube holders, gastric tube insertion systems, gastrostomy feeding tube holders, medical tubing retaining devices, stomach tube fixation devices and the like having various designs, configurations and structures have been disclosed in the prior art. For example, U.S. Pat. No. 5,304,145 to BLAIR discloses a gastrostomy tube holder arranged for ease of access and employment by a patient. The tube holder includes a belt member arranged for torso mounting relative to an individual having a cooperative support cover plate to secure the gastrostomy tube for subsequent use, with a slot directed through the support plate for access into the individual for use. This prior art patent does not disclose or teach the structure, configuration and design of the holder for a stomach insert feeding tube of the present invention.

U.S. Pat. No. 6,814,716 to BOUPHAVICHITH, et al. discloses a clip device for externally retaining a gastrostomy feeding tube against the stomach of a patient. The clip is a generally rectangular, unitary structure shaped to include a first end wall, a second end wall spaced apart from and extending parallel to the first end wall, a lower end wall extending perpendicularly between and interconnecting the first end wall and the second end wall, and an upper wall having a first end connected to the first end wall and a second end spaced apart from the second end wall. The medical tube has a distal end implanted in the patient. This prior art patent does not disclose or teach the structure, configuration and design of the holder for a stomach insert feeding tube of the present invention.

U.S. Pat. No. 5,271,745 to FENTRESS et al. discloses a retaining device for a medical tubing device for use on the stomach wall of a patient. The tubing retaining device includes a one-piece flexible band containing members to form a sling. The sling is of sufficient length to retain coiled medical tubing when affixed to the band. This tubing retaining device is used to retain tubing from in-dwelling devices such as catheters. This prior art patent does not disclose or teach the structure, configuration, and design of the holder for a stomach insert feeding tube of the present invention.

U.S. Pat. No. 4,834,712 to QUINN, et al. discloses a tube fixation device for the angular fixation of a delivery tube or a drainage tube at the point where the tube exits through the skin from a body cavity. The delivery tube or drainage tube may be used for delivery, extraction, or evacuation of fluids or gases through a surgically formed stomach opening in a patient. In the enteral feeding or delivery embodiment, an enteral feeding tube extends outwardly from the patient to support the components of the device. The components include a sleeve movable along the feeding tube and base elbow unit through which the feeding tube passes. The base elbow unit includes a subdermal portion, a sleeve engaging portion, and a generally cylindrical tube-engaging portion. The feeding tube is movable from a first substantially straight position to a second acutely angled position. In this second acutely angled position, the tube is immobilized by locking the engagement to the tube engaging portion. The tube fixation device includes an interlocking tab at one end of the sleeve, which tab enables locking engagement of the sleeve engaging portion of the base elbow unit when the tube is in its second acutely angled position. This prior art patent does not disclose or teach the structure, configuration and design of the holder for a stomach insert feeding tube of the present invention.

U.S. Pat. No. 5,358,488 to SURIYAPA discloses a gastronomy device to control gastronomy leakage that includes an elongated feeding tube having a first end for insertion through an opening in a patient's abdominal wall and a second end including a feeding inlet. An inflatable balloon is mounted near the first end and a leakage seal valve is connected adjacent to the balloon. The seal valve includes a flexible, elastomeric member to seal the opening in the patient and prevent leakage. This prior art patent does not disclose or teach the structure, configuration and design of the holder for a stomach insert feeding tube of the present invention.

None of the aforementioned prior art patents teach or disclose a stomach insert feeding tube holder having a substantially donut-shaped housing with a center opening for receiving an insert feeding tube therethrough; which minimizes chaffing caused by movement of the feeding tube at the stomach penetration point.

Accordingly, it is an object of the present invention to provide a stomach insert feeding tube holder that includes a substantially donut-shaped housing having a center opening for receiving an insert feeding tube therethrough, in order to minimize the chaffing caused by the feeding tube movement at the stomach penetration point on the patient.

Another object of the present invention is to provide a stomach insert feeding tube holder that eliminates stomach abrasions from the stomach section in contact with the tube holder caused by the lateral movement of the tube holder on the stomach.

Another object of the present invention is to provide a stomach insert feeding tube holder that lessens the chances of patient infection caused by movement of the tube holder on the stomach.

Another object of the present invention is to provide a stomach insert feeding tube holder that allows the individual patient a modicum of patient comfort when the holder is in an operational mode.

Another object of the present invention is to provide a stomach insert feeding tube holder that has a slot or channel for removing the feeding tube holder without the need for removing the feeding tube from the stomach wall of the patient.

Another object of the present invention is to provide a stomach insert feeding tube holder that is made of soft, durable and pliable plastic materials, such as medical grade silicone which can be sterilized for patient safety.

A further object of the present invention is to provide a stomach insert feeding tube holder that can be mass-produced in an automated and economical manner and is readily affordable by the user.

### SUMMARY OF THE INVENTION

In accordance with the present invention, there is a provided a holder for receiving a stomach insert feeding tube therein for use in the gastronomical feeding of a patient. The feeding tube holder includes a substantially donut-shaped housing having a passageway for receiving the feeding tube therethrough. The donut-shaped housing includes a channel connected to the passageway for allowing the removal of the feeding tube holder without the need for removing the feeding tube from the stomach wall of the patient. The donut-shaped housing also includes an outer flange member for contacting a pad positioned on the stomach surface of the patient. Additionally, the donut-shaped housing further includes an exterior circular side wall and an interior circular side wall being connected to each other by a top wall member. The exterior side wall has a greater height than the interior side wall; and the interior side wall is used for maintaining the passageway at an elevated position spaced above the stomach surface for maintaining the feeding tube at a fixed position above the stomach surface.

### BRIEF DESCRIPTION OF THE DRAWINGS

Further objects, features and advantages of the present invention will become apparent upon consideration of the following detailed description of the presently-preferred embodiment when taken in conjunction with the accompanying drawings, wherein:

FIG. 1 is a top perspective view of the stomach insert feeding tube holder of the preferred embodiment of the present invention showing a donut-shaped housing having a center opening for receiving an insert feeding tube therethrough;

FIG. 2 is a cross-sectional elevational view of the stomach insert feeding tube holder of the present invention, taken along lines 2-2 of FIG. 1 in the direction of the arrows, showing an outer lip for stomach contact, the donut-shaped housing, and the center opening;

FIG. 3 is a top plan view of the stomach insert feeding tube holder of the present invention showing the insert feeding tube within the center opening and in an operational mode on the patient's stomach;

FIG. 4 is a cross-sectional view of the stomach insert feeding tube holder of the present invention showing the holder in an operational mode on the patient's stomach; and

FIG. 5 is a top perspective view of the stomach insert feeding tube holder of the present invention showing the feeding tube holder being removed or reinserted about the feeding tube through a slot or channel when in an operational mode.

### DETAILED DESCRIPTION OF THE PREFERRED EMBODIMENT

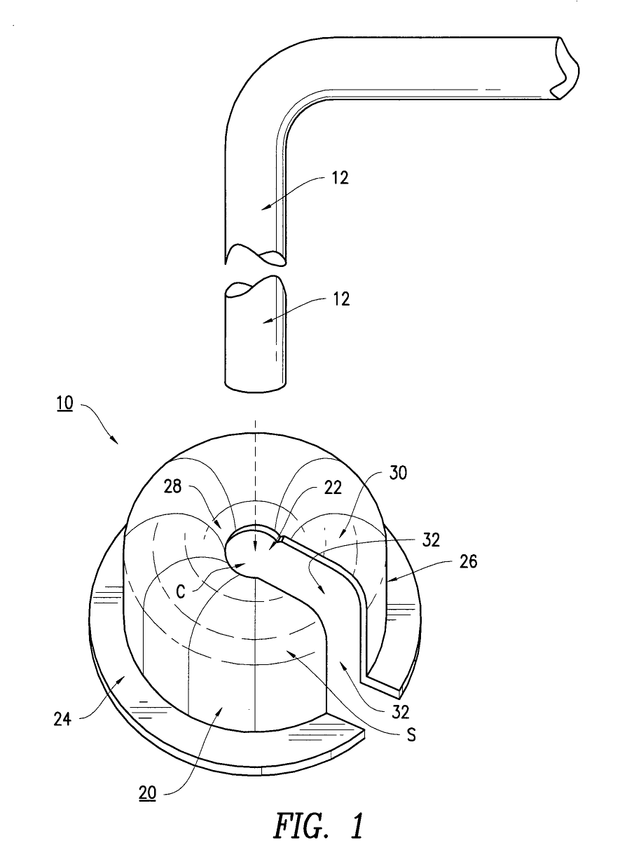

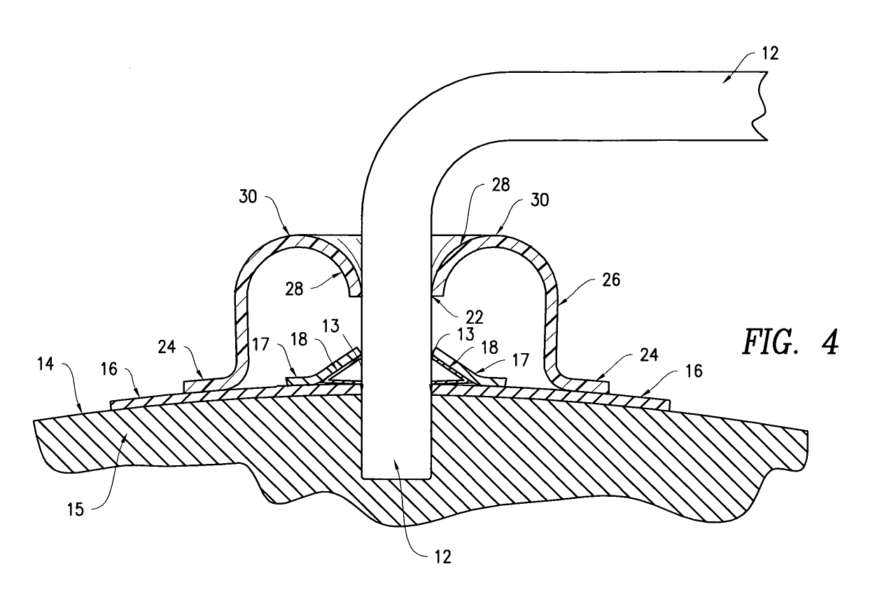

The stomach insert feeding tube holder 10 and its component parts of the preferred embodiment of the present invention are represented in detail by FIGS. 1 through 4 of the patent drawings. The feeding tube holder 10 is used in conjunction with a feeding tube 12 which is inserted within a center opening 22 of the holder 10 at a stomach penetration point 13 in the stomach wall surface 14. In this manner, the feeding tube 12 is inserted into the intestinal tract (not shown) or the interior stomach (not shown)of the patient 15, as shown in FIG. 4 of the drawings. The holder 10 for the stomach insert feeding tube 12 is made of soft durable and pliable plastic materials, such as medical grade silicone material S which is sterilizable for patient safety.

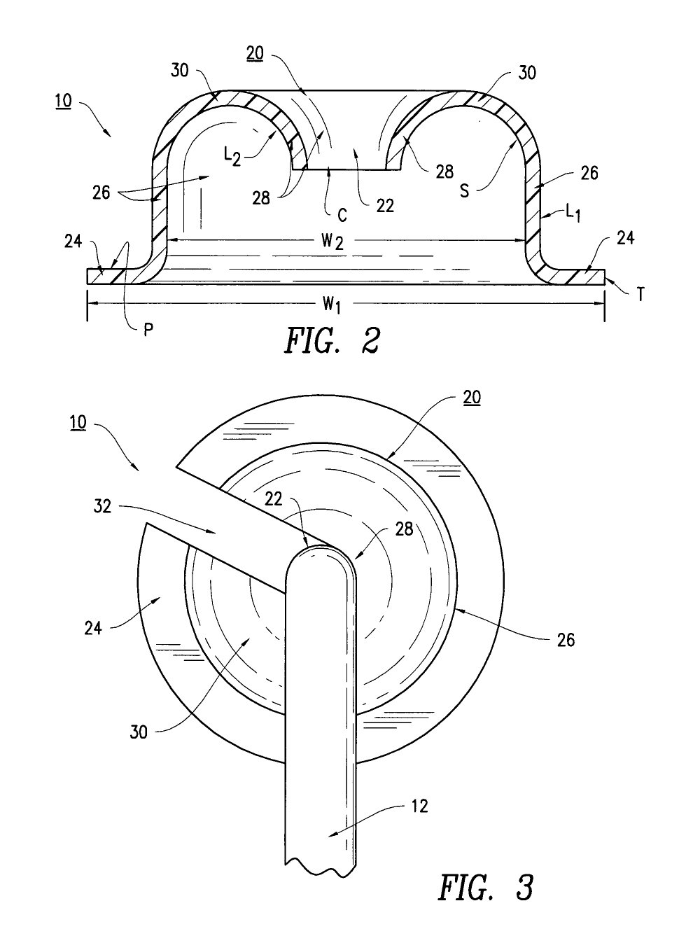

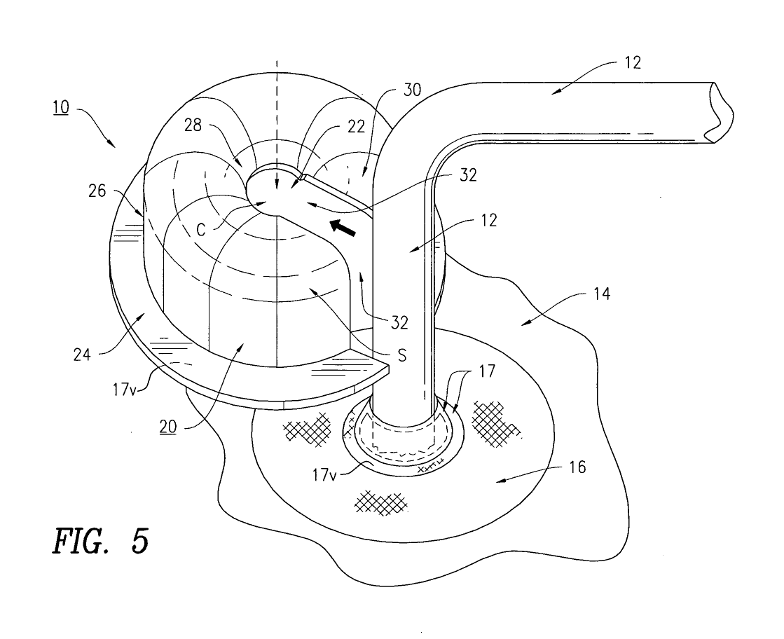

The holder 10 for the stomach insert feeding tube 12, as shown in FIGS. 1 and 2, includes a donut-shaped housing 20 having a center opening 22 for receiving the feeding tube 12 therethrough (see FIG. 4). The donut-shaped housing 20 includes an outer perimeter lip 24 for contacting a medical gauze pad 16 on the stomach wall surface 14 (see FIG. 4), adjacent to and surrounding the stomach penetration point 13. The donut-shaped housing 20 also includes a first exterior circumferential perimeter side wall 26 and a second interior circumferential perimeter side wall 28, such that side walls 26 and 28 are integrally connected to a top wall 30, as shown in FIG. 2 of the drawings. Side wall 26 is greater in height than side wall 28, L₁>L₂ as shown in FIG. 2. The holder 10 further includes a holding clip 18 for engaging and securing the feeding tube 12 in order to hold the feeding tube 12 in position relative to the stomach wall surface 14 (see FIG. 4). Additionally, the donut-shaped housing 20 includes a slot or channel 32 extending from the center opening 22 through the top wall 30 to the perimeter side wall 26 and to the outer perimeter lip 24, as shown in FIGS. 1 and 5 of the patent drawings. The slot or channel 32 allows the tube holder 10 to be removed and replaced around the feeding tube 12 for cleaning (see FIG. 5) without the need for removing the feeding tube 12 from the stomach wall 14 of patient 15.

The physical dimensions of the feeding tube holder 10 are as follows: The overall width diameter W₁ is 3.25 inches ± 1/32 of an inch, the interior diameter W₂ is 2.0 inches ± 1/32 of an inch, and the center opening C is 0.3125 inches ± 1/64 of an inch. The exterior side wall L₁ height is 2.0 inches ± 1/32 of an inch and the interior side wall L₂ height is 1.75 inches ± 1/32 of an inch. The outer perimeter lip P is 0.625 inches ± 1/32 of an inch. The wall thickness T is 0.05 inches ± 1/64 of an inch. The channel opening 32 is 0.30 inches ± 1/64 of an inch.

### OPERATION OF THE PRESENT INVENTION

As shown in FIGS. 1 to 4, the holder 10 for the stomach insert feeding tube 12 operates in the following manner: The physician, and/or nurse initially makes an incision in the stomach wall surface 14 for starting a stomach penetration point 13 for inserting the feeding tube 12 in a later step. The physician/nurse then places a medical gauze pad 16 having an opening over the stomach penetration point 13. Then the outer perimeter lip 24 is positioned on the medical gauze pad 16 surrounding the stomach penetration point 13. The outer perimeter lip 24 of holder 10 is then secured to the surrounding stomach wall surface 14 with medical tape 17 or Velcro™ 17v or other adhering means, as shown in FIGS. 4 and 5. The feeding tube 12, as shown in FIGS. 1, 3 and 4, is then inserted into the center opening 22 and stomach penetration point 13 for insertion into the intestinal tract (not shown). Then the feeding tube 12 and the holding clip 18 are secured together above the medical gauze pad 16 (see FIG. 4).

In performing the above procedure, the holder 10 minimizes chaffing caused by movement of the feeding tube 12 at the stomach penetration point 13. This eliminates abrasions and lessening the chance of stomach infections and also providing physical comfort to the patient.

If needed, the feeding tube holder 10 can be removed and replaced around the feeding tube 12 for cleaning by simply sliding the tube holder 10 away from the feeding tube 12 using the slot or channel 32 (see FIG. 5) without the need for removing the feeding tube 12 from the stomach wall 14 of patient 15.

### ADVANTAGES OF THE PRESENT INVENTION

Accordingly, an advantage of the present invention is that it provides for a stomach insert feeding tube holder that includes a substantially donut-shaped housing having a center opening for receiving an insert feeding tube therethrough, in order to minimize the chaffing caused by the feeding tube movement at the stomach penetration point on the patient.

Another advantage of the present invention is that it provides for a stomach insert feeding tube holder that eliminates stomach abrasions from the stomach section in contact with the tube holder caused by the lateral movement of the tube holder on the stomach.

Another advantage of the present invention is that it provides for a stomach insert feeding tube holder that lessens the chances of patient infection caused by movement of the tube holder on the stomach.

Another advantage of the present invention is that it provides for a stomach insert feeding tube holder that allows the individual patient a modicum of patient comfort when the holder is in an operational mode.

Another advantage of the present invention is that it provides for a stomach insert feeding tube holder that has a slot or channel for removing the feeding tube holder without the need for removing the feeding tube from the stomach wall of the patient.

Another advantage of the present invention is that it provides for a stomach insert feeding tube holder that is made of soft, durable and pliable plastic materials, such as medical grade silicone which can be sterilized for patient safety.

A further advantage of the present invention is that it provides for a stomach insert feeding tube holder that can be mass-produced in an automated and economical manner and is readily affordable by the user.

A latitude of modification, change and substitution is intended in the foregoing disclosure, and in some instances, some features of the invention will be employed without a corresponding use of other features. Accordingly, it is appropriate that the appended claims be construed broadly and in a manner consistent with the spirit and scope of the invention herein.

What is claimed is:

1. A holder for receiving therein a stomach insert feeding tube for use in gastronomical feeding of a patient, comprising:
a) a feeding tube holder including a housing;
b) said housing having a passageway for receiving the feeding tube therethrough;
c) said housing also including a channel connected to said passageway for allowing the removing of said feeding tube holder without the need for removing the feeding tube from the stomach wall of the patient;
d) said housing including an outer flange member for contacting a pad positioned on the stomach surface of the patient;
e) said housing further including an exterior circular side wall and an interior circular side wall being connected to each other by a top wall member; and
f) said exterior side wall having a greater height than said interior side wall; and wherein said interior side wall is used for maintaining said passageway at an elevated position spaced above the stomach surface for maintaining the feeding tube at a fixed position above the stomach surface.

2. A feeding tube holder in accordance with claim 1, wherein said passageway is centrally positioned within said housing and is adjacent to said interior side wall.

3. A feeding tube holder in accordance with claim 1, wherein said passageway is of a size for tightly engaging and holding the feeding tube in a fixed position relative to the stomach surface.

4. A feeding tube holder device in accordance with claim 1, further including a holding clip for engaging and securing the feeding tube in order to hold the feeding tube in position relative to the stomach surface.

5. A feeding tube holder in accordance with claim 1, wherein said housing is substantially donut-shaped having an interior compartment.

6. A feeding tube holder in accordance with claim 1, wherein said feeding tube holder is made of a soft, durable and pliable plastic material.

7. A feeding tube holder in accordance with claim 6, wherein said soft, durable and pliable plastic material is a medical grade silicone material.

8. A feeding tube holder in accordance with claim 7, wherein said medical grade silicone material is sterilizable for patient safety.

9. A feeding tube holder device in accordance with claim 1, wherein said housing has an overall diameter of 3.25 inches ± 1/32 of an inch and has an interior diameter of 2.0 inches ± 1/32 of an inch.

10. A feeding tube holder device in accordance with claim 1, wherein said passageway opening has a diameter of 0.3125 inches ± 1/64 of an inch.

11. A feeding tube holder device in accordance with claim 1, wherein said exterior side wall has a height of 2.0 inches ± 1/32 of an inch and said interior side wall has a height of 1.75 inches ± 1/32 of an inch.

12. A feeding tube holder device in accordance with claim 1, wherein said outer flange member has a width of 0.625 inches ± 1/32 of an inch.

13. A feeding tube holder device in accordance with claim 1, wherein said donut-shaped housing has a wall thickness of 0.05 inches ± 1/64 of an inch.

14. A feeding tube holder device in accordance with claim 1, wherein said channel has a width of 0.30 inches ± 1/32 of an inch.

15. A holder for receiving therein a stomach insert feeding tube for use in gastronomical feeding of a patient, comprising:
a) a feeding tube holder including a housing;
b) said housing having a passageway for receiving the feeding tube therethrough;
c) said housing also including a channel connected to said passageway for allowing the removing of said feeding tube holder without the need for removing the feeding tube from the stomach wall of the patient; and
d) said housing including an outer flange member for contacting a pad positioned on the stomach surface of the patient.

16. A feeding tube holder in accordance with claim 15, wherein said housing further includes an exterior circular side wall and an interior circular side wall being connected to each other by a top wall member.

17. A feeding tube holder in accordance with claim 16, wherein said exterior side wall having a greater height than said interior side wall; and wherein said interior side wall is used for maintaining said passageway at an elevated position spaced above the stomach surface for maintaining the feeding tube at a fixed position above the stomach surface.

18. A feeding tube holder in accordance with claim 15, wherein said passageway is centrally positioned with said housing and is adjacent to said interior side wall.

19. A feeding tube holder in accordance with claim 15, wherein said passageway is of a size for tightly engaging and holding the feeding tube in a fixed position relative to the stomach surface.

20. A feeding tube holder in accordance with claim 15, wherein said housing is substantially donut-shaped having an interior compartment.
